(19) United States
(12) Patent Application Publication
(10) Pub. No.: US 2018/0369596 A1
(43) Pub. Date: Dec. 27, 2018

(54) SYSTEMS AND METHODS FOR PROVIDING AN AUXILIARY ELECTRICAL CONNECTION ALONG AN ELECTRICAL STIMULATION SYSTEM

(71) Applicant: Boston Scientific Neuromodulation Corporation, Valencia, CA (US)

(72) Inventor: Jeffery Van Funderburk, Stevenson Ranch, CA (US)

(21) Appl. No.: 16/017,659

(22) Filed: Jun. 25, 2018

Related U.S. Application Data
(60) Provisional application No. 62/524,935, filed on Jun. 26, 2017.

(51) Int. Cl. A61N 1/372; A61N 1/36; A61N 1/05; A61N 1/378; A61N 1/08; H01R 24/58

(52) U.S. Cl. CPC A61N 1/37229; A61N 1/3606; A61N 1/0534; A61N 1/3727; H01R 2107/00; A61N 1/37241; A61N 1/08; H01R 24/58; A61N 1/3787

(57) ABSTRACT

An electrical stimulation lead or lead extension with a body having a proximal portion, a distal portion, and a longitudinal length. Distal contacts are disposed along the distal portion of the body; terminals are disposed along the proximal portion of the body; and conductors electrically couple the terminals to the distal contacts. An auxiliary terminal includes an electrically-conductive material and is disposed along the proximal portion of the body. An antenna is attached to the auxiliary terminal and extends along the longitudinal length of the electrical stimulation lead or lead extension no more than 10 cm.

### CROSS-REFERENCE TO RELATED APPLICATIONS

This application claims the benefit under 35 U.S.C. § 119(e) of U.S. Provisional Patent Application Ser. No. 62/524,935, filed Jun. 26, 2017, which is incorporated herein by reference.

### FIELD

The present invention is directed to the area of implantable electrical stimulation systems and methods of making and using the systems. The present invention is also directed to systems and methods for providing an auxiliary electrical contact within a control module of an electrical stimulation system suitable for forming an auxiliary electrical connection with a lead, as well as methods of making and using the leads, control modules, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator) and one or more stimulator electrodes. The one or more stimulator electrodes can be disposed along one or more leads, or along the control module, or both. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is an electrical stimulation lead or lead extension with a body having a proximal portion, a distal portion, and a longitudinal length. Distal contacts are disposed along the distal portion of the body; terminals are disposed along the proximal portion of the body; and conductors electrically couple the terminals to the distal contacts. An auxiliary terminal includes an electrically-conductive material and is disposed along the proximal portion of the body. An antenna is attached to the auxiliary terminal and extends along the longitudinal length of the electrical stimulation lead or lead extension no more than 10 cm.

In at least some embodiments, the antenna terminates within the body. In at least some embodiments, the antenna does not attach to any of the distal contacts. In at least some embodiments, the antenna is configured to receive data at frequencies no less than 1 GHz and no greater than 10 GHz. In at least some embodiments, the antenna extends along the longitudinal length of the electrical stimulation lead or lead extension no more than 5 cm.

In at least some embodiments, the distal contacts include lead-extension contacts disposed in a connector of a lead extension configured and arranged to couple with an electrical stimulation lead. In at least some embodiments, the distal contacts include electrodes disposed along an electrical stimulation lead.

In another embodiment, an electrical stimulation system includes the electrical stimulation lead or lead extension described above. A control module is coupleable with the electrical stimulation lead or lead extension. The control module includes a housing and an electronic subassembly disposed in the housing. The control module additionally includes a connector disposed in the housing and configured and arranged to receive a portion of the electrical stimulation lead or lead extension. The connector includes a connector lumen and connector contacts arranged along the connector lumen and electrically coupled to the electronic subassembly. The control module further includes an auxiliary connector contact disposed in the connector along the connector lumen and electrically coupled to the electronic subassembly. The auxiliary connector contact includes a retention assembly formed, at least in part, from an electrically-conductive material and configured and arranged to physically engage the auxiliary terminal of the lead or lead extension to facilitate retention of the received portion of the electrical stimulation lead or lead extension in the connector.

In at least some embodiments, a programmer is configured and arranged to communicate stimulation parameters to the electronic subassembly at a programming frequency, and the antenna is designed to receive the stimulation parameters from the programmer at the programming frequency.

In yet another embodiment, a control module of an electrical stimulation system includes a housing and an electronic subassembly disposed in the housing. The control module additionally includes a connector disposed in the housing and configured and arranged to receive a portion of a lead or lead extension. The connector includes a connector lumen and connector contacts arranged along the connector lumen and electrically coupled to the electronic subassembly. The control module further includes an auxiliary connector contact disposed in the connector along the connector lumen and electrically coupled to the electronic subassembly. The auxiliary connector contact includes a retention assembly formed, at least in part, from an electrically-conductive material and configured and arranged to physically engage the received portion of the lead or lead extension to facilitate retention of the received portion of the lead or lead extension in the connector.

In at least some embodiments, interconnect wires electrically couple the connector contacts to the electronic subassembly. In at least some embodiments, an auxiliary interconnect wire electrically couples the auxiliary connector contact to the electronic subassembly. In at least some embodiments, the auxiliary interconnect wire is shielded.

In still yet another embodiment, an electrical stimulation system includes the control module described above and a lead coupleable to the control module. The lead includes a lead body having a proximal portion, a distal portion, and a longitudinal length; electrodes disposed along the distal portion of the lead body; terminals disposed along the proximal portion of the lead body; conductors electrically coupling the terminals to the electrodes; an auxiliary terminal that includes an electrically-conductive material disposed along the proximal portion of the lead body and electrically coupled with the electronic subassembly; an auxiliary electrode disposed along the lead body; and an auxiliary conductor electrically coupling the auxiliary terminal to the auxiliary electrode.

In at least some embodiments, the electrical stimulation system further includes a lead extension configured and arranged to electrically couple the auxiliary terminal of the lead to the auxiliary connector contact of the control module.

In another embodiment, a method for stimulating patient tissue includes advancing a lead to a target stimulation location within a patient. The lead includes electrodes disposed along a distal portion of the lead; terminals disposed along a proximal portion of the lead; conductors electrically coupling the terminals to the electrodes; an auxiliary terminal with electrically-conductive material disposed along the proximal portion of the lead; and an auxiliary conductor attached to the auxiliary terminal and extending along a longitudinal length of the lead. The lead is coupled to the control module described above with the terminals electrically coupled to the connector contacts of the control module and the auxiliary terminal electrically coupled to the auxiliary connector contact of the control module. Patient tissue is stimulated using the electrodes.

In at least some embodiments, the method further includes communicating stimulation parameters, using a programmer, to the electronic subassembly of the control module at a programming frequency, where the auxiliary conductor functions as an antenna designed to receive the stimulation parameters from the programmer at the programming frequency and provide the received stimulation parameters to the electronic subassembly.

In at least some embodiments, the method further includes stimulating patient tissue using an auxiliary electrode disposed along the lead and electrically coupled to the auxiliary conductor.

In at least some embodiments, the method further includes sensing electrical activity at the target stimulation location using an auxiliary electrode disposed along the lead and electrically coupled to the auxiliary conductor.

In still yet another embodiment, a method for stimulating patient tissue includes providing the electrical stimulation system described above. The electrical stimulation lead or lead extension of the electrical stimulation system is implanted into a patient. The control module is implanted into the patient. The control module is coupled to the electrical stimulation lead or lead extension. Stimulation parameters are delivered to the control module through the antenna of the electrical stimulation lead or lead extension.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2 is a schematic side view of one embodiment of an electrical stimulation lead, according to the invention;

FIG. 3 is a schematic side view of one embodiment of a lead extension suitable for coupling with the electrical stimulation lead of FIG. 2, according to the invention;

FIG. 4 is a schematic side view of one embodiment of the lead of FIG. 2 coupled to the lead extension of FIG. 3, according to the invention;

FIG. 5 is a schematic side view of one embodiment of a control module suitable for receiving either the lead of FIG. 2 or the lead extension of FIG. 3, according to the invention;

FIG. 6 is a schematic side view of one embodiment of an elongated member retained by the control module of FIG. 5, according to the invention;

FIG. 7 is a schematic side view of one embodiment of a control module with an auxiliary connector-contact assembly for providing an auxiliary electrical connection between the control module and a retained elongated member, according to the invention;

FIG. 8 is a schematic side view of one embodiment of a lead suitable for being retained by the control module of FIG. 7, the lead including an auxiliary elongated-member conductor coupled to an auxiliary terminal, according to the invention;

FIG. 9 is a schematic side view of one embodiment of a lead extension suitable for being retained by the control module of FIG. 7, the lead extension including an auxiliary elongated-member conductor coupled to an auxiliary terminal, according to the invention;

FIG. 10 is a schematic side view of one embodiment of a lead suitable for being retained by the control module of FIG. 7, the lead including an auxiliary electrode coupled to an auxiliary terminal by an auxiliary elongated-member conductor, according to the invention;

FIG. 11 is a schematic side view of one embodiment of a lead extension with an auxiliary terminal suitable for facilitating coupling of the lead extension to the control module of FIG. 7, the lead extension also including a lead-extension auxiliary connector contact suitable for facilitating coupling of the lead extension to the lead of FIG. 10, the lead-extension auxiliary terminal electrically coupled to the lead-extension auxiliary connector contact by an auxiliary elongated-member conductor, according to the invention;

FIG. 12 is a schematic side view of one embodiment of the lead of FIG. 10 coupled to the lead extension of FIG. 12, according to the invention; and

FIG. 13 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to systems and methods for providing an auxiliary electrical contact within a control module of an electrical stimulation system suitable for forming an auxiliary electrical connection with a lead, as well as methods of making and using the leads, control modules, and electrical stimulation systems.

Suitable implantable electrical stimulation systems include, but are not limited to, a least one lead with one or more electrodes disposed on a distal portion of the lead and one or more terminals disposed on one or more proximal portions of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference. In the discussion below, a percutaneous lead will be exemplified, but it will be understood that the methods and systems described herein are also applicable to paddle leads and other leads.

A percutaneous lead for electrical stimulation (for example, deep brain, spinal cord, peripheral nerve, or cardiac-tissue stimulation) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes. For illustrative purposes, the leads are described herein relative to use for deep brain stimulation, but it will be understood that any of the leads can be used for applications other than deep brain stimulation, including spinal cord stimulation, peripheral nerve stimulation, dorsal root ganglion stimulation, sacral nerve stimulation, or stimulation of other nerves, muscles, and tissues.

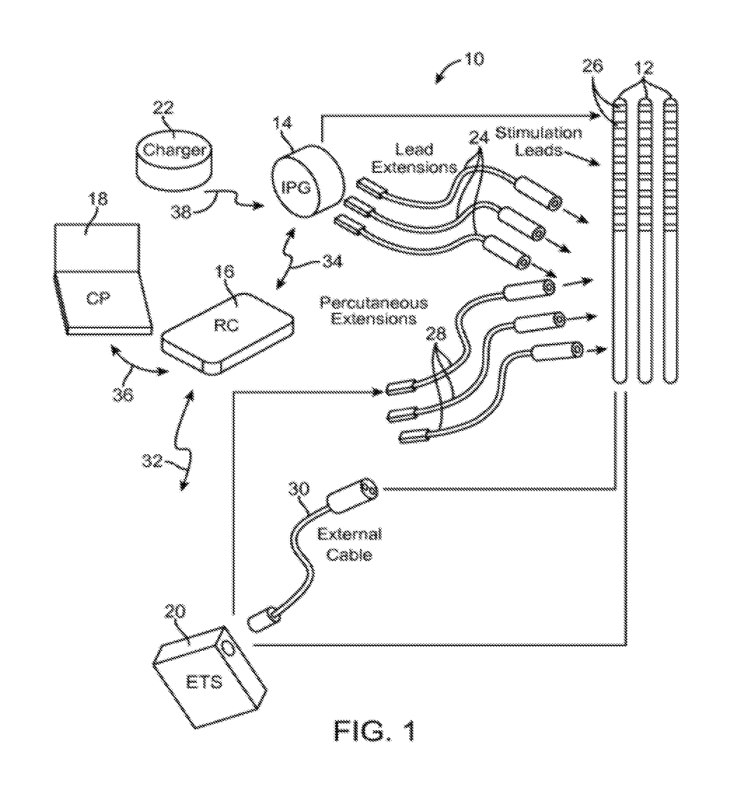

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22.

The IPG 14 is physically connected, optionally, via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's buttocks or abdominal cavity. The implantable pulse generator can have eight stimulation channels which may be independently programmable to control the magnitude of the current stimulus from each channel. In some embodiments, the implantable pulse generator can have more or fewer than eight stimulation channels (e.g., 4-, 6-, 16-, 32-, or more stimulation channels). The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and U.S. Pat. Nos. 7,761,165; 7,974,706; 8,175,710; 8,224,450; and 8,364,278; and U.S. Patent Application Publication No. 2007/0150036, as well as the other references cited above, all of which are incorporated by reference.

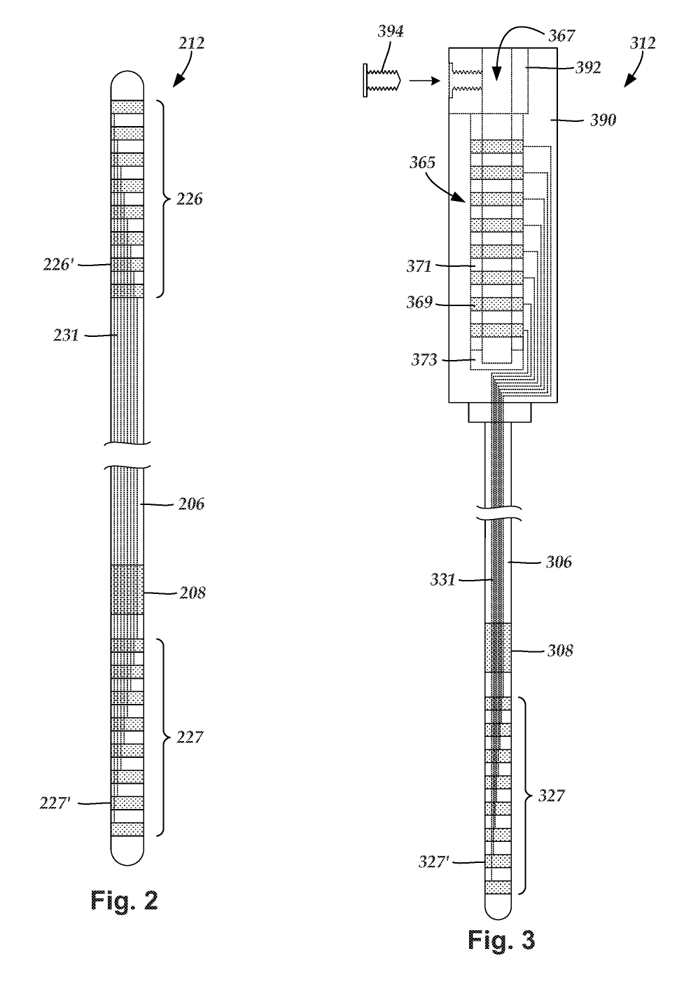

Turning to FIG. 2, one or more leads are configured for coupling with a control module. The term “control module” is used herein to describe a pulse generator (e.g., the IPG 14 or the ETS 20 of FIG. 1). Stimulation signals generated by the control module are emitted by electrodes of the lead(s) to stimulate patient tissue. The electrodes of the lead(s) are electrically coupled to terminals of the lead(s) that, in turn, are electrically coupleable with the control module. In some embodiments, the lead(s) couple(s) directly with the control module. In other embodiments, one or more intermediary devices (e.g., a lead extension, an adaptor, a splitter, or the like) are disposed between the lead(s) and the control module.

Percutaneous leads are described herein for clarity of illustration. It will be understood that paddle leads and cuff leads can be used in lieu of, or in addition to, percutaneous leads. The leads described herein include 8 electrodes (+1 auxiliary electrode in some embodiments). It will be understood that the leads could include any suitable number of electrodes. The leads described herein exclusively include ring electrodes. It will be understood that the leads can include a distal-tip electrode, or one or more segmented electrodes in lieu of, or in addition to one or more ring electrodes. Additionally, the term “elongated member” used herein includes leads (e.g., percutaneous, paddle, cuff, or the like), as well as intermediary devices (e.g., lead extensions, adaptors, splitters, or the like).

FIG. 2 shows, in schematic side view, one embodiment of a lead 212 suitable for implanting into a patient and providing electrical stimulation. In some embodiments, the lead 212 is coupled directly to a control module. In other embodiments, the lead 212 is coupled to the control module via one or more intermediary devices. In the illustrated embodiment, an array of electrodes 226, which includes electrode 226′, is disposed along a distal portion of a lead body 206 lead and an array of lead terminals 227, which includes lead terminal 227′, is disposed along a proximal portion of the lead body. Lead conductors, such as lead conductor 231, extend along a longitudinal length of the lead and electrically couple the array of electrodes 226 to the array lead terminals 227.

Conductors can extend along the longitudinal length of the lead within one or more lumens defined in the lead. In other instances, the conductors may extend along the lead within the lead body itself. The lead 212 includes an auxiliary terminal 208 disposed along the proximal portion of the body to facilitate coupling of the proximal portion of the lead to a connector. The connector may be disposed along a control module. Alternatively, the auxiliary terminal 208 can be used to facilitate coupling of the proximal portion of the lead to a connector of an intermediary device, such as a lead extension which, in turn, is coupled to a connector of a control module.

FIG. 3 shows, in schematic side view, one embodiment of a lead extension 312 suitable for implanting into a patient and coupling a lead, such as the lead 212, to a control module. The lead extension 312 includes a lead-extension body 306 having a distal portion and a proximal portion. A lead-extension connector 390 is disposed along the distal portion of the lead-extension body 306 and an array of lead-extension terminals 327, such as lead-extension terminal 327′, are disposed along the proximal portion of the lead-extension body 306.

The lead-extension connector 390 contains a lead-extension connector stack 365 that defines a connector lumen 367 configured to receive the proximal portion of an elongated member (e.g., lead 212). The lead-extension connector stack 365 includes lead-extension connector contacts, such as lead-extension connector contact 369, arranged along the connector lumen 367 and configured to electrically couple with terminals of the elongated member (e.g., lead 212) when the proximal portion of the elongated member is received by the lead-extension connector 390. The connector contacts are electrically isolated from one another by electrically-nonconductive spacers, such as spacer 371. The connector stack may also include an end stop 373 to promote alignment of the elongated-member terminals with the lead-extension connector contacts.

The lead-extension connector 390 further includes a retention assembly for facilitating retention of the proximal portion of the elongated member (e.g., lead 212) when the proximal portion of the elongated member is received by the lead-extension connector 390. In the illustrated embodiment, the retention assembly includes a lead-extension retention block 392. The lead-extension retention block 392 is positioned to align with the auxiliary terminal (208 in FIG. 2) of the elongated member when the elongated member is received by the lead-extension connector 390. In the illustrated embodiment, the retention assembly further includes a retaining member (e.g., a set screw, a pin, or the like) 394 for pressing the auxiliary terminal of the inserted elongated member against the retention block to retain inserted elongated member within the lead-extension connector 390.

Lead-extension conductors, such as lead-extension conductor 331, extend along a longitudinal length of the lead extension and electrically couple the lead-extension connector contacts to the array of lead-extension terminals 327. The lead-extension conductors can extend along the longitudinal length of the lead-extension body within one or more lumens defined in the lead extension. In other instances, the lead-extension conductors may extend along the lead extension within the lead-extension body itself. The lead extension 312 includes an auxiliary terminal 308 disposed along the proximal portion of the lead-extension body to facilitate coupling of the proximal portion of the lead extension to a connector, such as a control-module connector, another lead-extension connector, or the like.

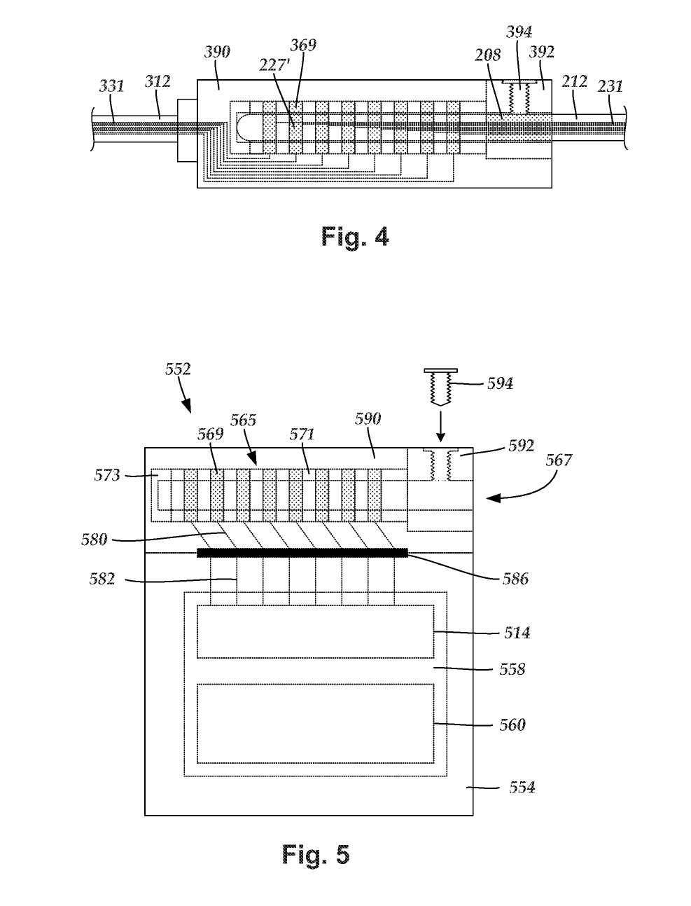

FIG. 4 shows, in schematic side view, one embodiment of the lead 212 received by the lead-extension connector 390. In the illustrated embodiment, the lead terminals 227, such as lead terminal 227′, are aligned with the lead-extension connector contacts, such as lead-extension connector contact 369. Accordingly, the lead conductors 231 are electrically coupled with the lead-extension conductors 331. Additionally, in the illustrated embodiment the lead auxiliary terminal 208 is aligned with the lead-extension retention block 392 and the retaining member 394 is pressing the lead auxiliary terminal 208 against the lead-extension retention block to retain the lead 212 within the lead-extension connector 390.

FIG. 5 shows, in schematic cross-sectional side view, one embodiment of a control module 552 suitable for coupling with an elongated member (e.g., the lead 212, the lead extension 312, or other intermediary device). The control module 552 includes a housing having a sealed portion 554 that houses an electronic subassembly 558 with a pulse generator 514 and, optionally, a power supply 560.

The housing further includes an unsealed portion that includes a connector 590 configured to receive an elongated device (e.g., the lead 212, the lead extension 312, or other intermediary device). Optionally, the connector 590 is positioned along an outer surface of the sealed portion of the housing. The connector 590 contains a connector stack 565 that defines a connector lumen 567 configured to receive the proximal portion of the elongated member. The connector stack 565 includes an array of connector contacts, such as connector contact 569, arranged along the connector lumen 567 and configured to electrically couple with terminals of the elongated member when the proximal portion of the elongated member is received by the connector 590. The connector contacts are electrically isolated from one another by electrically-nonconductive spacers, such as spacer 571. The connector stack may also include an end stop 573 to promote alignment of the elongated-member terminals with the connector contacts.

Feedthrough interconnects, such as feedthrough interconnect 582, are electrically coupled to the electrical subassembly 558 and extend within the sealed portion of the housing to a feedthrough interface 586 disposed along an interface between the sealed and unsealed portions of the housing. The connector contacts are electrically coupled to interconnect wires, such as interconnect wire 580, that extend along the unsealed portion of the housing and electrically couple the connector contacts to the feedthrough interconnects at the feedthrough interface 586. In some embodiments, the connector 590 is positioned along an outer surface of the sealed housing over the feedthrough interface 586. In other embodiments, the connector 590 is disposed at least partially within an outer surface of the sealed housing.

The connector 590 further includes a retention assembly for facilitating retention of the proximal portion of the elongated member when the proximal portion of the elongated member is received by the control module 552. In the illustrated embodiment, the retention assembly includes a retention block 592. The retention block 592 is positioned to align with the auxiliary terminal (208 in FIG. 2; 308 in FIG. 3) of the elongated member when the elongated member is received by the control module 552. In the illustrated embodiment, the retention assembly further includes a retaining member (e.g., a set screw, a pin, or the like) 594 for pressing the auxiliary terminal of the inserted elongated member against the retention block to retain inserted elongated member within the control module 552.

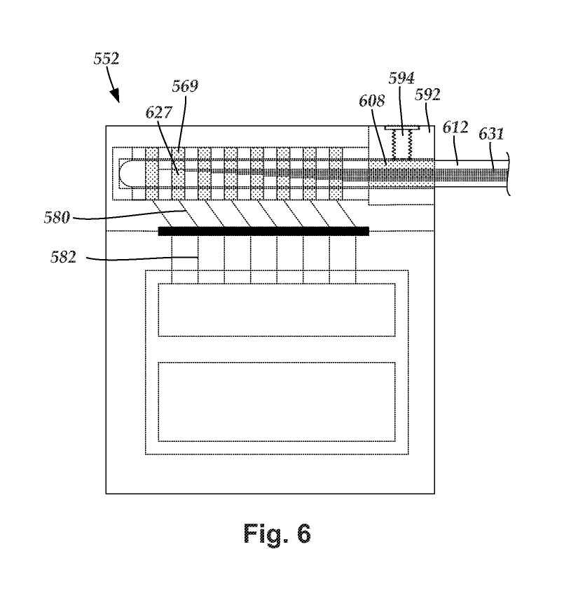

FIG. 6 shows, in schematic side view, one embodiment of an elongated member 612 (e.g., the lead 212, the lead extension 312, or other intermediary device) received by the control module 552. In the illustrated embodiment, the elongated-member terminals, such as elongated-member terminal 627, are aligned with the connector contacts, such as connector contact 569. Accordingly, the elongated-member conductors 631 are electrically coupled with the interconnect wires 580 and feedthrough interconnects 582. Additionally, in the illustrated embodiment an auxiliary terminal 608 disposed along the elongated member 612 is aligned with the retention block 592 and the retaining member 594 is pressing the elongated-member auxiliary terminal 608 against the retention block to retain the elongated member 612 within the control module 552.

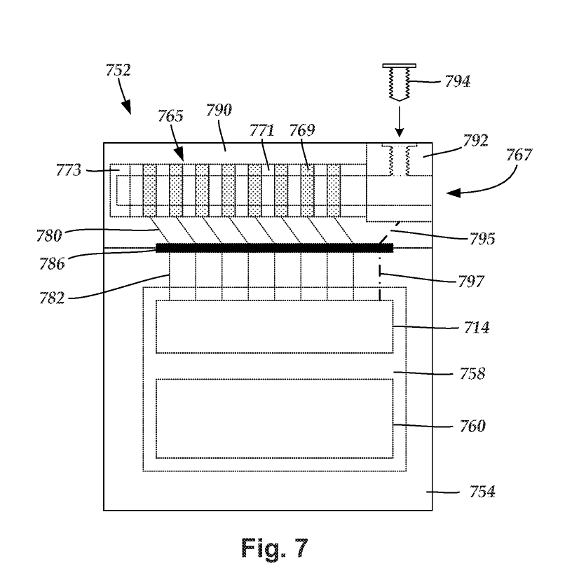

Turning to FIG. 7, as herein described an auxiliary connector-contact assembly forms an auxiliary connector contact suitable for creating an auxiliary electrical connection between the pulse generator and an elongated member (e.g., the lead 212, the lead extension 312, or other intermediary device) when the elongated member is received by the control module. The auxiliary electrical connection is separate from, and additional to, the above-described electrical connections between the connector contacts and terminals of an elongated member. The auxiliary connector contact is formed using the retention block of the control module and the auxiliary terminal of an elongated member, both of which can be formed, at least in part, from electrically-conductive materials.

FIG. 7 shows, in schematic cross-sectional side view, one embodiment of a control module 752 suitable for coupling with an elongated member (e.g., the lead 212, the lead extension 312, or other intermediary device). The control module 752 includes a housing having a sealed portion 754 that houses an electronic subassembly 758 with a pulse generator 714 and, optionally, a power supply 760.

The housing further includes an unsealed portion that includes a connector 790 configured to receive an elongated device (e.g., the lead 212, the lead extension 312, or other intermediary device). Optionally, the connector 790 is positioned along an outer surface of the sealed portion of the housing. The connector 790 contains a connector stack 765 that defines a connector lumen 767 configured to receive the proximal portion of the elongated member. The connector stack 765 includes an array of connector contacts, such as connector contact 769, arranged along the connector lumen 767 and configured to electrically couple with terminals of the elongated member when the proximal portion of the elongated member is received by the connector 790. The connector contacts are electrically isolated from one another by electrically-nonconductive spacers, such as spacer 771. The connector stack may also include an end stop 773 to promote alignment of the elongated-member terminals with the connector contacts.

Feedthrough interconnects, such as feedthrough interconnect 782, are electrically coupled to the electrical subassembly 758 and extend within the sealed portion of the housing to a feedthrough interface 786 disposed along an interface between the sealed and unsealed portions of the housing. The connector contacts are electrically coupled to interconnect wires, such as interconnect wire 780, that extend along the unsealed portion of the housing and electrically couple the connector contacts to the feedthrough interconnects at the feedthrough interface 786. In some embodiments, the connector 790 is positioned along an outer surface of the sealed housing over the feedthrough interface 786. In other embodiments, the connector 790 is disposed at least partially within an outer surface of the sealed housing.

The connector 790 further includes a retention assembly for facilitating retention of the proximal portion of the elongated member when the proximal portion of the elongated member is received by the control module 752. In the illustrated embodiment, the retention assembly includes an auxiliary connector contact 792. The auxiliary connector contact 792 is positioned to align with the auxiliary terminal of an elongated member when the elongated member is received by the control module 752. In the illustrated embodiment, the retention assembly further includes a retaining member (e.g., a set screw, a pin, or the like) 794 for pressing the auxiliary terminal of the inserted elongated member against the auxiliary connector contact to retain inserted elongated member within the control module 752.

An auxiliary connector-contact assembly forms an auxiliary electrical connection between the electronic subassembly and the retention assembly. In the illustrated embodiment, the retention assembly includes the auxiliary connector contact 792, which functions both to retain an inserted elongated member, as well as to form an auxiliary connector contact. In some embodiments, the auxiliary connector contact 792 is formed, at least in part, from an electrically-conductive material.

In at least some embodiment, the auxiliary connector-contact assembly includes an auxiliary interconnect wire coupling the retention assembly to the feedthrough interface. In the illustrated embodiment, the auxiliary connector contact 792 is electrically coupled to an auxiliary interconnect wire 795 suitable for electrically coupling the auxiliary connector contact 792 to the feedthrough interface 786. The auxiliary interconnect wire 795 may, optionally, be shielded. In at least some embodiments, the auxiliary interconnect wire 795 is formed as a coaxial cable. In at least some embodiments, the auxiliary interconnect wire 795 is suitable for receiving or transmitting (or both) data at frequencies of 1 GHz, 1.5 GHz, 2 GHz, 2.5 GHz, 3 GHz, 4 GHz, 5 GHz, 6 GHz, 7 GHz, 8 GHz, 9 GHz, 10 GHz, or greater. In at least some embodiments, the auxiliary interconnect wire 795 is suitable for receiving or transmitting (or both) data at frequencies no less than 1 GHz and no greater than 10 GHz. In at least some embodiments, the auxiliary interconnect wire 795 electrically couples the auxiliary connector contact 792 to an auxiliary feedthrough interconnect 797 at the feedthrough interface 786. The auxiliary feedthrough interconnect 797 extends within the sealed portion of the housing and electrically couples to the electrical subassembly 758.

It will be understood that the auxiliary interconnect wire can additionally, or alternatively, be used for other purposes that utilize different frequencies than the one described above. In at least some embodiments, the auxiliary interconnect wire is suitable for receiving or transmitting (or both) data at frequencies of 100 kHz, 105 kHz, 110 kHz, 115 kHz, 120 kHz, 125 kHz, 130 kHz, 135 kHz, or greater. In at least some embodiments, the auxiliary interconnect wire is suitable for receiving or transmitting (or both) data at frequencies of 370 MHz, 380 MHz, 390 MHz, 400 MHz, 410 MHz, 420 MHz, 430 MHz, 440 MHz, or greater.

In the illustrated embodiments, the control module includes a single connector stack configured to receive a single elongated member, and a single retention assembly configured to retain the single elongated member. In at least some embodiments, the control module includes multiple connector stacks (e.g., 2, 3, 4, or more connector stacks). In at least some embodiments, each of the connector stacks is configured to receive a different elongated member. In at least some embodiments, each of the multiple elongated members is retained by one or more retention assemblies.

In at least some embodiments, the control module includes multiple auxiliary connector-contact assemblies. In at least some embodiments, each of the multiple auxiliary connector-contact assemblies includes a different retention assembly from the remaining auxiliary connector-contact assemblies. In at least some embodiments, the number of auxiliary connector-contact assemblies is equal to the number of retention assemblies. In at least some embodiments, at least two of the multiple auxiliary connector-contact assemblies use the same retention assembly.

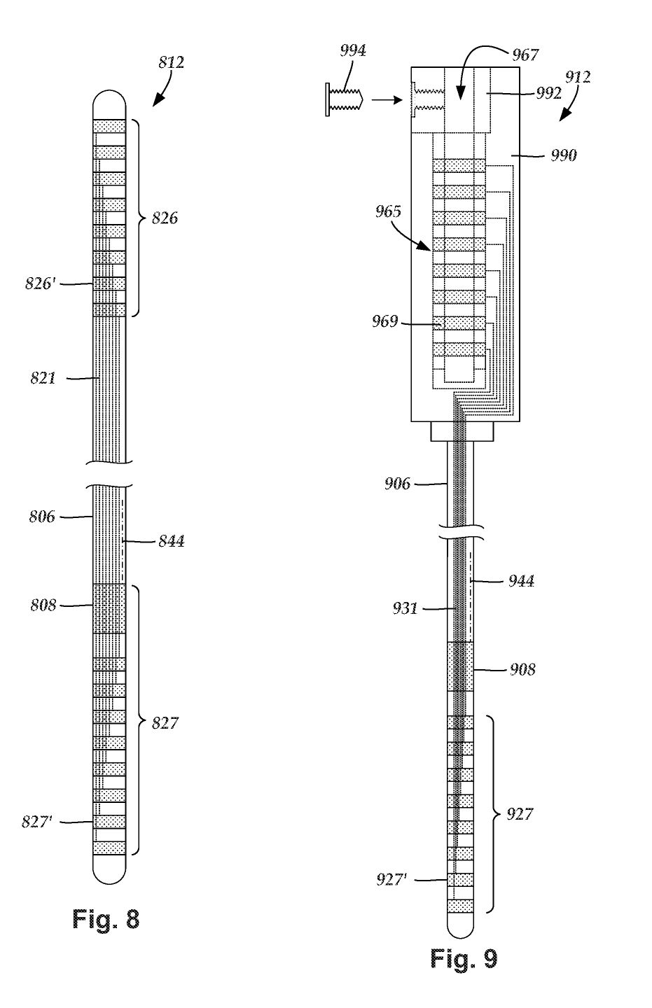

Turning to FIGS. 8-9, in at least some embodiments an elongated member insertable into the control module includes at least one auxiliary elongated-member conductor that is electrically coupled to the auxiliary terminal. Accordingly, the auxiliary elongated-member conductor is coupleable to the electronic subassembly when the elongated member is received by the control module and the auxiliary terminal of the elongated member is retained by the connector assembly.

In at least some embodiments, the auxiliary elongated-member conductor extends along the longitudinal length of an elongated member by an amount sufficient to function as an antenna, such as a Bluetooth antenna or other antenna. In at least some embodiments, the antenna terminates within the body of the elongated member (e.g., the lead or lead extension). In at least some embodiments, the antenna does not attach to distal contacts of an elongated member (e.g., electrodes of leads, contacts of lead extensions).

At least some electrical stimulation systems include an antenna within the control module. It may be an advantage to move the antenna to a location external to the control module to provide additional space within the control module for other components, or to enable the control module to be made smaller. Additionally, it may improve reception to have an antenna extend away from potential interference generated by metal and electronics within the control module.

The antenna can be extended along a longitudinal length of the elongated member by any suitable amount. In at least some embodiments, the antenna extends along a longitudinal length of the elongated member by no more than 50 cm, 40 cm, 30 cm, 20 cm, 15 cm, 10 cm, 9 cm, 8 cm, 7 cm, 6 cm, 5 cm, 4 cm, 3 cm, 2 cm, 1 cm. In at least some embodiments, the antenna extends along a longitudinal length of the elongated member by no more than 50%, 40%, 30%, 20%, 10%, 5% of the longitudinal length. In at least some embodiments, the antenna includes a coil. In at least some embodiments, the antenna is suitable for receiving or transmitting (or both) data at frequencies of 1 GHz, 1.5 GHz, 2 GHz, 2.5 GHz, 3 GHz, 4 GHz, 5 GHz, 6 GHz, 7 GHz, 8 GHz, 9 GHz, 10 GHz, or greater. In at least some embodiments, the antenna is suitable for receiving or transmitting (or both) data at frequencies no less than 1 GHz and no greater than 10 GHz.

It will be understood that the antenna can additionally, or alternatively, be used for other purposes that utilize different frequencies than the one described above. In at least some embodiments, the antenna wire is suitable for receiving or transmitting (or both) data at frequencies of 100 kHz, 105 kHz, 110 kHz, 115 kHz, 120 kHz, 125 kHz, 130 kHz, 135 kHz, or greater. In at least some embodiments, the antenna is suitable for receiving or transmitting (or both) data at frequencies of 370 MHz, 380 MHz, 390 MHz, 400 MHz, 410 MHz, 420 MHz, 430 MHz, 440 MHz, or greater.

In some embodiments, the antenna is disposed in a lead coupleable to the control module 752. FIG. 8 shows, in schematic side view, one embodiment of a lead 812 suitable for implanting into a patient and coupling to the control module 752 for providing electrical stimulation. In the illustrated embodiment, an array of electrodes 826, which includes electrode 826′, is disposed along a distal portion of a lead body 806 lead and an array of lead terminals 827, which includes lead terminal 827′ is disposed along a proximal portion of the lead body. Lead conductors, such as lead conductor 831, extend along a longitudinal length of the lead and electrically couple the array of electrodes 826 to the array lead terminals 827.

The lead conductors 821 can extend along the longitudinal length of the lead within one or more lumens defined in the lead. In other instances, the lead conductors may extend along the lead within the lead body itself. The lead 812 includes an auxiliary terminal 808 disposed along the proximal portion of the body to facilitate coupling of the proximal portion of the lead to a connector, such as a control-module connector or a lead-extension connector. The auxiliary terminal 808 is formed, at least in part, from electrically-conductive material.

The lead 812 further includes an antenna 844 electrically coupled to the auxiliary terminal 808 and extending along the lead 812. In some embodiments, the antenna 844 extends along the longitudinal length of the lead within one or more lumens defined in the lead. In at least some embodiments, the antenna 844 extends along a lumen within which at least one of the lead conductors 831 also extends. In other embodiments, the antenna 844 extends along the lead 812 within the lead body 806 itself.

In some embodiments, the antenna is disposed in a lead extension coupleable to the control module 752. FIG. 9 shows, in schematic side view, one embodiment of a lead extension 912 suitable for implanting into a patient and coupling to the control module 752. In the illustrated embodiment, a lead-extension connector 990 is disposed along a distal portion of a lead-extension body 906 and an array of lead-extension terminals 927, such as lead-extension terminal 927′, are disposed along an opposing proximal portion of the lead-extension body 906. An auxiliary terminal 908 is also disposed along the proximal portion of the lead-extension body to facilitate coupling of the proximal portion of the lead extension to a connector, such as a control-module connector, another lead-extension connector, or the like. The auxiliary terminal 908 is formed, at least in part, from electrically-conductive material.

The lead-extension connector 990 contains a lead-extension connector stack 965 that defines a connector lumen 967 configured to receive the proximal portion of another elongated member (e.g., a lead). The lead-extension connector stack 965 includes lead-extension connector contacts, such as lead-extension connector contact 969, arranged along the connector lumen 967 and configured to electrically couple with terminals of an elongated member (e.g., a lead) when the proximal portion of the elongated member is received by the lead-extension connector stack 965.

The lead-extension connector 990 further includes a lead-extension retention assembly for facilitating retention of the proximal portion of an elongated member when the proximal portion of the elongated member is received by the lead-extension connector 990. In the illustrated embodiment, the lead-extension retention assembly includes a lead-extension retention assembly that includes a lead-extension auxiliary connector contact 992. The lead-extension auxiliary connector contact 992 is positioned to align with the auxiliary terminal of the elongated member when the elongated member is received by the lead-extension connector 990. In the illustrated embodiment, a retaining member (e.g., a set screw, a pin, or the like) 994 is used for pressing the auxiliary terminal of the inserted elongated member against the auxiliary connector contact to retain inserted elongated member within the control module 990.

Lead-extension conductors, such as lead-extension conductor 931, extend along a longitudinal length of the lead extension and electrically couple the lead-extension connector contacts to the array of lead-extension terminals 927. Lead-extension conductors can extend along the longitudinal length of the lead-extension body within one or more lumens defined in the lead extension. In other instances, the lead-extension conductors may extend along the lead extension within the lead-extension body itself.

The lead extension 912 further includes an antenna 944 electrically coupled to the auxiliary terminal 908 and extending along the lead extension 912. In some embodiments, the antenna 944 extends along the longitudinal length of the lead extension within one or more lumens defined in the lead extension. In at least some embodiments, the antenna 944 extends along a lumen within which at least one of the lead conductors 931 also extends. In other embodiments, the antenna 944 extends along the lead extension 912 within the lead-extension body 906 itself.

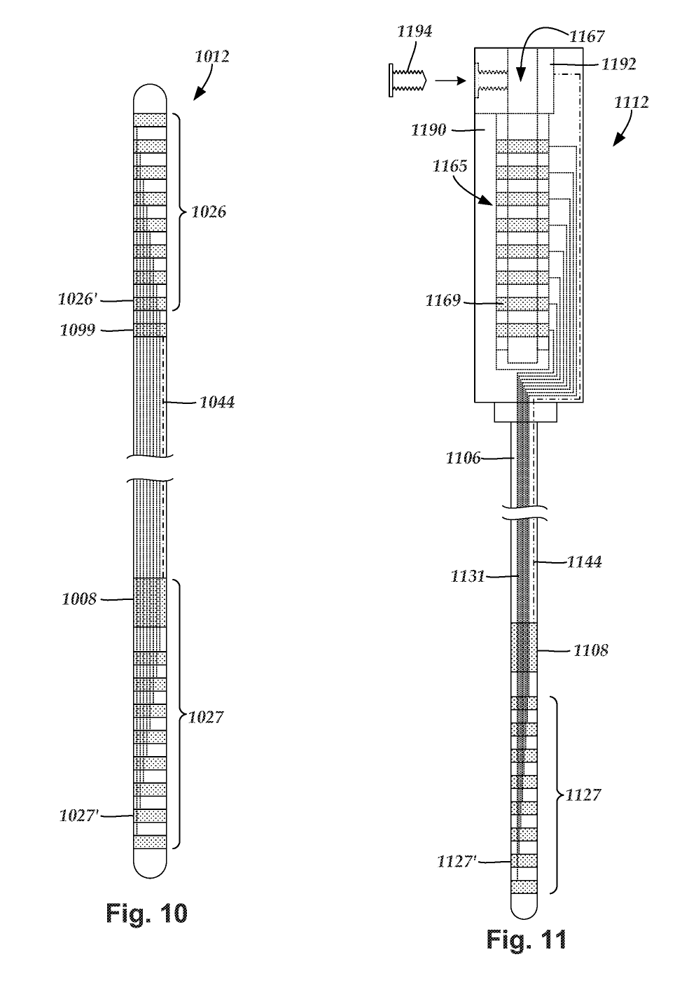

Turning to FIGS. 10-11, in at least some embodiments the auxiliary elongated-member conductor is electrically coupled to an auxiliary electrode disposed along a lead. In some embodiments, the lead is coupled directly to the control module. In other embodiments, the lead is coupled indirectly to the control module via one or more intermediary devices, such as a lead extension.

FIG. 10 shows, in schematic side view, one embodiment of a lead 1012 suitable for implanting into a patient and coupling to the control module 752 for providing electrical stimulation. In the illustrated embodiment, an array of electrodes 1026, which includes electrode 1026′, is disposed along a distal portion of a lead body 1006 lead and an array of lead terminals 1027, which includes lead terminal 1027′ is disposed along a proximal portion of the lead body.

Lead conductors, such as lead conductor 1031, extend along a longitudinal length of the lead and electrically couple the array of electrodes 1026 to the array lead terminals 1027. The lead 1012 includes an auxiliary terminal 1008 disposed along the proximal portion of the body to facilitate coupling of the proximal portion of the lead to a connector, such as a control-module connector or a lead-extension connector. The auxiliary terminal 1008 is formed, at least in part, from an electrically-conductive material. The auxiliary terminal 1008 is formed, at least in part, from one or more metals, alloys, or composites.

The lead 1012 further includes an auxiliary elongated-member conductor 1044 electrically coupling the auxiliary terminal 1008 to an auxiliary electrode 1099. The auxiliary electrode 1099 can be configured to function as a sensing electrode, a stimulation electrode, or both. The auxiliary electrode 1099 can be disposed at any suitable location along the longitudinal length of the lead. In the illustrated embodiment, the auxiliary electrode 1099 is disposed along the distal portion of the lead 1012 in proximity to the array of electrodes 1026. The auxiliary electrode 1099 could be any suitable type of electrode (e.g., a ring electrode, segmented electrode, distal-tip electrode, or the like). In the illustrated embodiment, the auxiliary electrode 1099 is a ring electrode.

In at least some embodiments, the lead 1012 is coupled to the control module 752 via one or more intermediary devices, such as a lead extension. FIG. 11 shows, in schematic side view, one embodiment of a lead extension 1112 suitable for implanting into a patient and coupling the control module 752 to the lead 1012. In the illustrated embodiment, a lead-extension connector 1190 is disposed along a distal portion of a lead-extension body 1106, and an array of lead-extension terminals 1127, such as lead-extension terminal 1127′, are disposed along an opposing proximal portion of the lead-extension body 1106.

A lead-extension auxiliary terminal 1108 is also disposed along the proximal portion of the lead-extension body to facilitate coupling of the proximal portion of the lead extension 1112 to a connector, such as the control-module connector, or another connector of an intermediary device (e.g., another lead-extension connector, or the like). The lead-extension auxiliary terminal 1108 is formed, at least in part, from an electrically-conductive material. The lead-extension auxiliary terminal 1108 is formed, at least in part, from one or more metals, alloys, or composites.

The lead-extension connector 1190 contains a lead-extension connector stack 1165 that defines a connector lumen 1167 configured to receive the proximal portion of another elongated member (e.g., the lead 1012). The lead-extension connector stack 1165 includes lead-extension connector contacts, such as lead-extension connector contact 1169, arranged along the connector lumen 1167 and configured to electrically couple with terminals of an elongated member (e.g., the lead 1012) when the proximal portion of the elongated member is received by the lead-extension connector stack 1165.

The lead-extension connector stack 1165 further includes a lead-extension retention assembly for facilitating retention of the proximal portion of an elongated member when the proximal portion of the elongated member is received by the lead-extension connector 1190. In the illustrated embodiment, the lead-extension retention assembly includes a lead-extension auxiliary connector contact 1192. The lead-extension auxiliary connector contact 1192 is positioned to align with the auxiliary terminal of the elongated member when the elongated member is received by the lead-extension connector 1190. In the illustrated embodiment, a retaining member (e.g., a set screw, a pin, or the like) 1194 is used for pressing the auxiliary terminal of the inserted elongated member against the auxiliary connector contact to retain inserted elongated member within the lead-extension connector 1190.

Lead-extension conductors, such as lead-extension conductor 1131, extend along a longitudinal length of the lead extension and electrically couple the lead-extension connector contacts to the array of lead-extension terminals 1127. The lead extension 1112 further includes an auxiliary elongated-member conductor 1144 electrically coupling the lead-extension auxiliary terminal 1108 to the lead-extension auxiliary connector contact 1192. In some embodiments, the lead-extension conductors and auxiliary elongated-member conductor extend along the longitudinal length of the lead-extension body within one or more lumens defined in the lead extension. In other embodiments, the lead-extension conductors and auxiliary elongated-member conductor may extend along the lead extension within the lead-extension body itself.

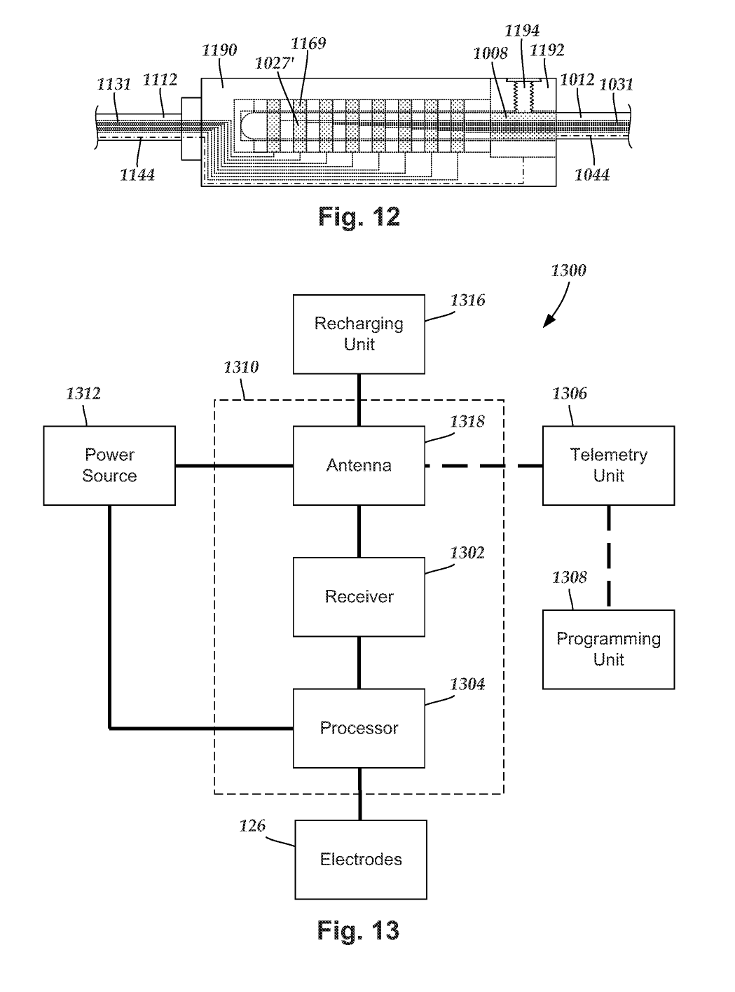

FIG. 12 shows, in schematic side view, one embodiment of the lead 1012 received by the lead-extension connector 1190. In the illustrated embodiment, the lead terminals, such as lead terminal 1027′, are aligned with the lead-extension connector contacts, such as lead-extension connector contact 1169. Accordingly, the lead conductors 1031 are electrically coupled with the lead-extension conductors 1131.

Additionally, in the illustrated embodiment the lead auxiliary terminal 1008 is aligned with the lead-extension auxiliary connector contact 1192 and the retaining member 1194 is pressing the lead auxiliary terminal 1008 against the lead-extension auxiliary connector contact to retain the lead 1012 within the lead-extension connector 1190. Accordingly, the elongated-member conductor 1144 of the lead extension 1112 is electrically coupled to the elongated-member conductor 1044 of the lead 1012, thereby enabling the auxiliary electrode (1099 in FIG. 10) to electrically couple to the electronic subassembly of the control module.

FIG. 13 is a schematic overview of one embodiment of components of an electrical stimulation system 1300 including an electronic subassembly 1310 disposed within a control module (see e.g., 752 in FIG. 7). The electronic subassembly 1310 may include one or more components of the IPG. It will be understood that the electrical stimulation system can include more, fewer, or different components and can have a variety of different configurations including those configurations disclosed in the stimulator references cited herein.

Some of the components (for example, a power source 1312, an antenna 1318, a receiver 1302, and a processor 1304) of the electrical stimulation system can be positioned on one or more circuit boards or similar carriers within a sealed housing of an implantable pulse generator (see e.g., 14 in FIG. 1), if desired. Any power source 1312 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1318 or a secondary antenna. In at least some embodiments, the antenna 1318 (or the secondary antenna) is implemented using the auxiliary electrically-conductive conductor. The external power source can be in a device that is mounted on the skin of the user or in a unit that is provided near the user on a permanent or periodic basis.

If the power source 1312 is a rechargeable battery, the battery may be recharged using the optional antenna 1318, if desired. Power can be provided to the battery for recharging by inductively coupling the battery through the antenna to a recharging unit 1316 external to the user. Examples of such arrangements can be found in the references identified above. The electronic subassembly 1310 and, optionally, the power source 1312 can be disposed within a control module (e.g., the IPG 14 or the ETS 20 of FIG. 1).

In one embodiment, electrical stimulation signals are emitted by the electrodes 126 on the paddle or lead body to stimulate nerve fibers, muscle fibers, or other body tissues near the electrical stimulation system. The processor 1304 is generally included to control the timing and electrical characteristics of the electrical stimulation system. For example, the processor 1304 can, if desired, control one or more of the timing, frequency, strength, duration, and waveform of the pulses. In addition, the processor 1304 can select which electrodes can be used to provide stimulation, if desired. In some embodiments, the processor 1304 selects which electrode(s) are cathodes and which electrode(s) are anodes. In some embodiments, the processor 1304 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1308 that, for example, allows modification of pulse characteristics. In the illustrated embodiment, the processor 1304 is coupled to a receiver 1302 which, in turn, is coupled to the optional antenna 1318. This allows the processor 1304 to receive instructions from an external source to, for example, direct the pulse characteristics and the selection of electrodes, if desired.

In one embodiment, the antenna 1318 is capable of receiving signals (e.g., RF signals) from an external telemetry unit 1306 which is programmed by the programming unit 1308. The programming unit 1308 can be external to, or part of, the telemetry unit 1306. The telemetry unit 1306 can be a device that is worn on the skin of the user or can be carried by the user and can have a form similar to a pager, cellular phone, or remote control, if desired. As another alternative, the telemetry unit 1306 may not be worn or carried by the user but may only be available at a home station or at a clinician's office. The programming unit 1308 can be any unit that can provide information to the telemetry unit 1306 for transmission to the electrical stimulation system 1300. The programming unit 1308 can be part of the telemetry unit 1306 or can provide signals or information to the telemetry unit 1306 via a wireless or wired connection. One example of a suitable programming unit is a computer operated by the user or clinician to send signals to the telemetry unit 1306.

The signals sent to the processor 1304 via the antenna 1318 and the receiver 1302 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1300 to cease operation, to start operation, to start charging the battery, or to stop charging the battery. In other embodiments, the stimulation system does not include the antenna 1318 or receiver 1302 and the processor 1304 operates as programmed.

Optionally, the electrical stimulation system 1300 may include a transmitter (not shown) coupled to the processor 1304 and the antenna 1318 for transmitting signals back to the telemetry unit 1306 or another unit capable of receiving the signals. For example, the electrical stimulation system 1300 may transmit signals indicating whether the electrical stimulation system 1300 is operating properly or not or indicating when the battery needs to be charged or the level of charge remaining in the battery. The processor 1304 may also be capable of transmitting information about the pulse characteristics so that a user or clinician can determine or verify the characteristics.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation lead or lead extension, comprising:
a body having a proximal portion, a distal portion, and a longitudinal length;
a plurality of distal contacts disposed along the distal portion of the body;
a plurality of terminals disposed along the proximal portion of the body;
a plurality of conductors electrically coupling the plurality of terminals to the plurality of distal contacts;
an auxiliary terminal comprising an electrically-conductive material and disposed along the proximal portion of the body; and
an antenna attached to the auxiliary terminal and extending along the longitudinal length of the body by no more than 10 cm.

2. The electrical stimulation lead or lead extension of claim 1, wherein the antenna terminates within the body.

3. The electrical stimulation lead or lead extension of claim 1, wherein the antenna does not attach to any of the plurality of distal contacts.

4. The electrical stimulation lead or lead extension of claim 1, wherein the antenna is configured to receive data at frequencies no less than 1 GHz and no greater than 10 GHz.

5. The electrical stimulation lead or lead extension of claim 1, wherein the antenna extends along the longitudinal length of the body by no more than 5 cm.

6. The electrical stimulation lead or lead extension of claim 1, wherein the plurality of distal contacts comprises lead-extension contacts disposed in a connector of a lead extension configured and arranged to couple with an electrical stimulation lead.

7. The electrical stimulation lead or lead extension of claim 1, wherein the plurality of distal contacts comprises electrodes disposed along an electrical stimulation lead.

8. An electrical stimulation system comprising:
the electrical stimulation lead or lead extension of claim 1; and
a control module coupleable with the electrical stimulation lead or lead extension, the control module comprising
a housing,
an electronic subassembly disposed in the housing,
a connector disposed in the housing and configured and arranged to receive a portion of the electrical stimulation lead or lead extension, the connector comprising a connector lumen and a plurality of connector contacts arranged along the connector lumen and electrically coupled to the electronic subassembly, and
an auxiliary connector contact disposed in the connector along the connector lumen and electrically coupled to the electronic subassembly, the auxiliary connector contact comprising a retention assembly formed, at least in part, from an electrically-conductive material and configured and arranged to physically engage the auxiliary terminal of the lead or lead extension to facilitate retention of the received portion of the electrical stimulation lead or lead extension in the connector.

9. The electrical stimulation system of claim 8, further comprising a programmer configured and arranged to communicate stimulation parameters to the electronic subassembly at a programming frequency, wherein the antenna is designed to receive the stimulation parameters from the programmer at the programming frequency.

10. A control module of an electrical stimulation system, the control module comprising:
a housing;
an electronic subassembly disposed in the housing;
a connector disposed in the housing and configured and arranged to receive a portion of a lead or lead extension, the connector comprising a connector lumen and a plurality of connector contacts arranged along the connector lumen and electrically coupled to the electronic subassembly; and
an auxiliary connector contact disposed in the connector along the connector lumen and electrically coupled to the electronic subassembly, the auxiliary connector contact comprising a retention assembly formed, at least in part, from an electrically-conductive material and configured and arranged to physically engage the received portion of the lead or lead extension to facilitate retention of the received portion of the lead or lead extension in the connector.

11. The control module of claim 10, further comprising a plurality of interconnect wires electrically coupling the plurality of connector contacts to the electronic subassembly.

12. The control module of claim 10, further comprising an auxiliary interconnect wire electrically coupling the auxiliary connector contact to the electronic subassembly.

13. The control module of claim 12, wherein the auxiliary interconnect wire is shielded.

14. An electrical stimulation system comprising:
the control module of claim 10;
a lead coupleable to the control module, the lead comprising
a lead body having a proximal portion, a distal portion, and a longitudinal length;
a plurality of electrodes disposed along the distal portion of the lead body;
a plurality of terminals disposed along the proximal portion of the lead body;
a plurality of conductors electrically coupling the plurality of terminals to the plurality of electrodes;
an auxiliary terminal comprising an electrically-conductive material and disposed along the proximal portion of the lead body, the auxiliary terminal configured and arranged to electrically couple with the electronic subassembly;
an auxiliary electrode disposed along the lead body; and
an auxiliary conductor electrically coupling the auxiliary terminal to the auxiliary electrode.

15. The electrical stimulation system of claim 14, further comprising a lead extension configured and arranged to electrically couple the auxiliary terminal of the lead to the auxiliary connector contact of the control module.

16. A method for stimulating patient tissue, the method comprising:
advancing a lead to a target stimulation location within a patient, the lead comprising a plurality of electrodes disposed along a distal portion of the lead, a plurality of terminals disposed along a proximal portion of the lead, a plurality of conductors electrically coupling the plurality of terminals to the plurality of electrodes, an auxiliary terminal comprising electrically-conductive material and disposed along the proximal portion of the lead, and an auxiliary conductor attached to the auxiliary terminal and extending along a longitudinal length of the lead;
coupling the lead to the control module claim 9 with the plurality of terminals electrically coupled to the plurality of connector contacts of the control module and the auxiliary terminal electrically coupled to the auxiliary connector contact of the control module; and
stimulating patient tissue using the plurality of electrodes.

17. The method of claim 16, further comprising communicating stimulation parameters, using a programmer, to the electronic subassembly of the control module at a programming frequency, wherein the auxiliary conductor functions as an antenna designed to receive the stimulation parameters from the programmer at the programming frequency and provide the received stimulation parameters to the electronic subassembly.

18. The method of claim 16, further comprising stimulating patient tissue using an auxiliary electrode disposed along the lead and electrically coupled to the auxiliary conductor.

19. The method of claim 16, further comprising sensing electrical activity at the target stimulation location using an auxiliary electrode disposed along the lead and electrically coupled to the auxiliary conductor.

20. A method for stimulating patient tissue, the method comprising:
providing the electrical stimulation system of claim 8;
implanting the electrical stimulation lead or lead extension of the electrical stimulation system into a patient;
implanting the control module into the patient;
coupling the control module to the electrical stimulation lead or lead extension; and
delivering stimulation parameters to the control module through the antenna of the electrical stimulation lead or lead extension.
